(19) United States
(12) Patent Application Publication
(10) Pub. No.: US 2021/0299162 A1
(43) Pub. Date: Sep. 30, 2021

(54) MOLECULAR HYDROGEN-CONTAINING COMPOSITION FOR PREVENTION AND/OR IMPROVEMENT OF ENCEPHALITIS AND/OR MENINGITIS AND SYMPTOM ASSOCIATED WITH THE ENCEPHALITIS AND/OR MENINGITIS

(71) Applicant: MiZ Company Limited, Kanagawa (JP)

(72) Inventors: Fumitake SATOH, Kanagawa (JP); Bunpei SATO, Kanagawa (JP); Yusuke ICHIKAWA, Kanagawa (JP); Shinichi HIRANO, Kanagawa (JP); Ryosuke KUROKAWA, Kanagawa (JP)

(73) Assignee: MiZ Company Limited, Kanagawa (JP)

(21) Appl. No.: 17/206,820

(22) Filed: Mar. 19, 2021

(30) Foreign Application Priority Data
Mar. 24, 2020 (JP) 2020-070639

(51) Int. Cl. A61K 33/00; A61P 29/00; A61K 9/00; C01B 3/00

(52) U.S. Cl. CPC A61K 33/00; C01B 3/00; A61K 9/007; A61P 29/00

(57) ABSTRACT

The present invention provides a composition for prevention and/or improvement of encephalitis and/or meningitis. More specifically, the present invention provides a composition for prevention and/or improvement of encephalitis and/or meningitis and a symptom associated with the encephalitis and/or meningitis in a subject, comprising molecular hydrogen as an active ingredient.

### RELATED APPLICATIONS

This application claims priority to Japanese Patent Application No. 2020-070639, filed on Mar. 24, 2020, the entire content of which is incorporated herein by reference.

### BACKGROUND

### 1. Technical Field

The present invention provides a molecular hydrogen-containing composition for prevention and/or improvement of encephalitis and/or meningitis and symptoms associated with the encephalitis and/or meningitis in subjects.

### 2. Description of the Related Art

Meningitis is a disease in which inflammation occurs in the meninges, which are layers covering the brain, and encephalitis is a disease in which inflammation occurs in the brain itself. Treatment methods and drugs used for meningitis and encephalitis vary depending on the types of the diseases. Many of the therapeutic agents cannot pass the blood-brain barrier and therefore cannot reduce intracerebral inflammation directly. Many patients with novel coronavirus-infected pneumonia, which originates in the outbreak in Wuhan in 2019, have been reported to have meningitis concurrently, leading to death from severe disturbance of consciousness and the like, but no treatment method has been established.

Hydrogen, the active ingredient of the present invention, has anti-oxidative reactivity, which reduces oxidative stress caused by reactive oxygen species, and is known to have an improving effect against various diseases such as refractory cancer and a respiratory disease (Malcolm Dole, F. Ray Wilson, William P. Fife; Science, New Series, Vol. 190, No. 4210 (Oct. 10, 1975), pp. 152-154; Japanese Patent No. 6628449). MiZ Company Limited proposes a theory that hydrogen is the most effective means to selectively eliminate hydroxyl radicals produced in a mitochondrion because a hydrogen molecule can easily reach the inside of the mitochondrion. However, there are no precedent of the effect of hydrogen to prevent and/or improve encephalitis and/or meningitis and a symptom associated with the encephalitis and/or meningitis having been documented so far.

Prevention and/or improvement of encephalitis and/or meningitis and a symptom associated with the encephalitis and/or meningitis would make it possible to achieve not only relief of distress and improvement of quality of life for patients, but also early recovery of, for example, patients with severe novel coronavirus pneumonia, and reducing the mortality. As described above, few components or substances are known to be useful for prevention and/or improvement of encephalitis and/or meningitis and a symptom associated with the encephalitis and/or meningitis.

Under such circumstances, an object of the present invention is to promote prevention and/or improvement of encephalitis and/or meningitis and a symptom associated with the encephalitis and/or meningitis by using molecular hydrogen.

### SUMMARY

That is, the present invention encompasses the following characteristics:

(1) A composition for prevention and/or improvement of encephalitis and/or meningitis and a symptom associated with the encephalitis and/or meningitis in a subject, comprising molecular hydrogen as an active ingredient.

(2) The composition according to (1), wherein the encephalitis and/or meningitis is one or more diseases selected from the group consisting of bacterial meningitis, tuberculous meningitis, fungal meningitis, viral meningitis, and herpes encephalitis.

(3) The composition according to (1) or (2), wherein the symptom associated with the encephalitis and/or meningitis is a symptom selected from the group consisting of headache, pyrexia, disturbance of consciousness, stiffness of the neck, vomiting, visual impairment, convulsion, memory disturbance, personality change, numbness, and abnormal behavior.

(4) The composition according to any one of (1) to (3), which is a liquid or a gas comprising the molecular hydrogen.

(5) The composition according to (4), wherein the liquid comprising the molecular hydrogen has a hydrogen concentration of 1 to 10 ppm.

(6) The composition according to (4), wherein the gas comprising the molecular hydrogen has a hydrogen concentration of higher than zero (0) and not higher than 18.5% by volume.

(7) The composition according to any one of (1) to (6), wherein the subject is a mammalian including a human.

(8) The composition according to any one of (1) to (7), which is produced by using a hydrogen gas generating apparatus, a hydrogen water generating apparatus, or a hydrogen gas adding apparatus.

The present invention can prevent and/or improve encephalitis and/or meningitis and a symptom associated with the encephalitis and/or meningitis in subjects. Additionally, refractory sepsis can also be improved by administering the composition according to the present invention to a subject.

### DETAILED DESCRIPTION OF THE PREFERRED EMBODIMENTS

The present invention will be described in more detail below.

### 1. Composition for Prevention and/or Improvement of Encephalitis and/or Meningitis and a Symptom Associated with the Encephalitis and/or Meningitis

The present invention provides a composition for prevention and/or improvement of encephalitis and/or meningitis and a symptom associated with the encephalitis and/or meningitis, comprising molecular hydrogen as an active ingredient.

In the present specification, the term “encephalitis” is a collective term for inflammatory diseases of the brain including herpes encephalitis.

In the present specification, the term “meninges” refers to membranes that exist between the skull bone and the brain to protect the brain by covering it. The meninges consist of three layers of dura mater, arachnoid mater, and pia mater from the outside.

In the present specification, the term “meningitis” refers to inflammation caused by infection with bacteria or virus, and examples thereof include bacterial meningitis, tuberculous meningitis, fungal meningitis, and viral meningitis as well as meningitis due to a spread of cancerous inflammation.

In the present specification, the term “a symptom associated with the encephalitis and/or meningitis” refers to a symptom such as headache, pyrexia, disturbance of consciousness, stiffness of the neck, vomiting, visual impairment, convulsion, memory disturbance, personality change, numbness, and abnormal behavior.

In the present specification, the term “a refractory genetic disorder” refers to a genetic disorder which is impossible or difficult to improve or resolve with current medicine or any drugs manufactured and marketed in the pharmaceutical industry, and for which treatment methods have not been established because of low evidence.

In the present specification, the term “subject” includes mammalians such as primates including humans, pet animals such as dogs and cats, and ornamental animals such as zoo animals. Preferred subjects are humans.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas. The concentration of hydrogen gas in a gas containing molecular hydrogen (i.e., the composition of the present invention) is higher than zero (0) and not higher than 18.5% by volume, for example, 0.5% to 18.5% by volume, preferably 1% to 10% by volume, for example, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, and the like. In the present invention, higher hydrogen gas concentrations (but below the explosion limit) or higher daily hydrogen doses tend to be associated with greater effects of promoting prevention and/or improvement (e.g., suppression or alleviation) of a genetic disorder.

Because hydrogen is a flammable and explosive gas, it is preferable to add hydrogen to the composition of the present invention under conditions safe for subjects such as humans and administer the mixture to subjects to prevent and/or improve a genetic disorder.

When a gas other than hydrogen gas is air, the air concentration is in the range of, for example, 81.5% to 99.5% by volume.

When a gas other than hydrogen gas is a gas containing oxygen gas, the oxygen gas concentration is in the rage of, for example, 21% to 99.5% by volume.

As another main gas, for example, nitrogen gas can be further added.

In a usual hydrogen gas inhalation therapy, an effect of improving a disease (cancer) is observed only when a hydrogen gas is used at a high concentration of 66% or 99%. In the present invention, however, it is preferable to add hydrogen to the composition of the present invention under safe conditions for subjects such as humans and administer it to a subject, and a sufficient effect of improving encephalitis and/or meningitis and a symptom associated with the encephalitis and/or meningitis can be exhibited even at low hydrogen concentrations of higher than 0 (zero) and not higher than 18.5%.

The liquids containing molecular hydrogen are specifically aqueous liquids containing a dissolved hydrogen gas. Examples of the aqueous liquids used herein include, but are not limited to, water (e.g., purified water, sterilized water), physiological saline, buffer solutions (e.g., buffer solutions of pH 4 to 7.4), drip infusion solutions, fluid infusion solutions, injection solutions, and drinks (e.g., tea drinks such as green tea and black tea, fruit juice, green juice, vegetable juice). Examples of the hydrogen concentration in a liquid containing molecular hydrogen include, but are not limited to, 1 to 10 ppm, preferably 1.2 to 9 ppm, for example, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, and 5 to 7 ppm.

In the present invention, higher concentrations of dissolved hydrogen (but below the explosion limit) or higher daily hydrogen doses tend to be associated with greater effects of preventing and/or improving encephalitis and/or meningitis and a symptom associated with the encephalitis and/or meningitis.

A gas or a liquid containing molecular hydrogen is formulated to provide a predetermined hydrogen gas concentration and then with the same, for example, a pressure-resistant container (e.g., a stainless cylinder, an aluminum can, a pressure-resistant plastic bottle [e.g., a pressure-resistant PET bottle] and a plastic bag preferably having the inside laminated with an aluminum film, or an aluminum bag) is filled. Aluminum has the property of unlikely allowing hydrogen molecules to pass therethrough. Alternatively, a gas containing molecular hydrogen or a liquid containing molecular hydrogen may be produced in situ before use by using an apparatus such as a hydrogen gas generating apparatus, a hydrogen water generating apparatus, or a hydrogen gas adding apparatus such as a known or commercially available hydrogen gas supply apparatus (an apparatus for generating a gas containing molecular hydrogen), a hydrogen adding device (an apparatus for hydrogen water generation), or a non-destructive hydrogen adding apparatus (e.g., an apparatus for non-destructively adding hydrogen gas into a bag for a biocompatible solution such as a drip infusion solution).

The hydrogen gas supply apparatus enables hydrogen gas generated from a reaction of a hydrogen generating agent (e.g., metallic aluminum, magnesium hydride) and water to be mixed with a diluent gas (e.g., air, oxygen) in a predetermined ratio (refer to Japanese Patent No. 5228142, etc.). Or, the hydrogen gas supply apparatus mixes hydrogen gas generated utilizing electrolysis of water with a diluent gas such as oxygen or air (refer to Japanese Patent No. 5502973, Japanese Patent No. 5900688, etc.). Thus, a gas containing molecular hydrogen at a hydrogen concentration in the range of, for example, 0.5% to 18.5% by volume can be prepared.

The hydrogen adding device is an apparatus that generates hydrogen by using a hydrogen generating agent and a pH modifier and dissolving the hydrogen in a biocompatible solution such as water (refer to Japanese Patent No. 4756102, Japanese Patent No. 4652479, Japanese Patent No. 4950352, Japanese Patent No. 6159462, Japanese Patent No. 6170605, Japanese Patent Laid-open No. 2017-104842, etc.). Examples of a mixture of a hydrogen generating agent and a pH modifier include metallic magnesium and a strongly acidic ion exchange resin or an organic acid (e.g., malic acid, citric acid) and a metallic aluminum powder and a calcium hydroxide powder. With these mixtures, a liquid containing molecular hydrogen at a dissolved hydrogen concentration of, for example, approximately 1 to 10 ppm can be prepared.

The non-destructive hydrogen adding apparatus is an apparatus or a device that adds hydrogen gas to a commercially available biocompatible solution such as a drip infusion solution (e.g., enclosed in a hydrogen-permeable plastic bag such as a polyethylene bag) from the outside of a package and is commercially available from, for example, MiZ Company Limited (http://www.e-miz.co.jp/technology.html). This apparatus can dissolve hydrogen in a biocompatible solution aseptically until the equilibrium concentration is reached, by immersing a bag containing the biocompatible solution in saturated hydrogen water, so that hydrogen is permeated into the bag. The apparatus is composed of, for example, an electrolytic bath and a water bath, and water in the water bath is circulated in the electrolytic bath and the water bath to generate hydrogen by electrolysis. Or, a simplified, disposable device can be used for a similar purpose (refer to Japanese Patent Laid-open No. 2016-112562, etc.). This device has a biocompatible solution-containing plastic bag (a hydrogen-permeable bag, for example, a polyethylene bag) and a hydrogen generating agent (e.g., metallic calcium, metallic magnesium/cation exchange resin) incorporated in an aluminum bag, and the hydrogen generating agent is wrapped with, for example, a non-woven fabric (e.g., steam-permeable non-woven fabric). Hydrogen generated by wetting the hydrogen generating agent wrapped with a non-woven fabric with a small amount of water, such as a steam, is dissolved in a biocompatible solution non-destructively and aseptically.

Or, a purified hydrogen gas cylinder, a purified oxygen gas cylinder, or a purified air cylinder may be provided to produce a gas or a liquid containing molecular hydrogen which is adjusted to provide a predetermined hydrogen concentration or a predetermined oxygen or air concentration.

A gas containing molecular hydrogen or a liquid containing molecular hydrogen (e.g., water [e.g., purified water, sterilized water], physiological saline, drip infusion solution) prepared using the above-mentioned apparatuses or devices can be administered orally or parenterally to subjects with encephalitis and/or meningitis and a symptom associated with the encephalitis and/or meningitis.

Other embodiments of the composition of the present invention include dosage forms (e.g., tablets, capsules) prepared to be orally administered to (or ingested by) subjects, which contain a hydrogen generating agent that enables hydrogen to be generated in the gastrointestinal tract. The hydrogen generating agent preferably comprises, for example, components approved as food or food additives.

When the composition of the present invention comprises molecular hydrogen as an active ingredient, examples of the method of administering the composition to subjects include administration by inhalation, suction or the like. For example, transpulmonary administration is preferred. When a liquid containing molecular hydrogen is contained as an active ingredient, oral or intravenous administration (including drip infusion) is preferred. When a gas is inhaled, the gas is inhaled from the mouth or the nose via a nasal cannula or a mask-like device covering the mouth and the nose, transported to the lungs, and delivered to the whole body by blood.

The liquid containing molecular hydrogen to be orally administered may be administered to subjects as a cooled liquid or a liquid stored at room temperature. Hydrogen is dissolved in water at a concentration of approximately 1.6 ppm (1.6 mg/L) at room temperature and under a normal pressure, and the difference in solubility due to temperature is known to be relatively small. Or, when a liquid containing molecular hydrogen is, for example, in the form of a drip infusion solution or an injection solution containing hydrogen gas prepared using the above-described non-destructive hydrogen adding apparatus, the liquid may be administered to subjects by parenteral routes, such as intravenous or intraarterial administration.

One dose or multiple doses (e.g., two to three doses) per day of a gas containing molecular hydrogen at the above-mentioned hydrogen concentrations or a liquid containing molecular hydrogen at the above-mentioned dissolved hydrogen concentrations can be administered to humans for a period of one week to three months or longer, for example, one week to six months or longer (e.g., one year or longer, two years or longer). When a gas containing molecular hydrogen is administered, the gas is preferably inhaled for at least 30 minutes per dose. Because the improving effect becomes higher with a longer inhalation time, the gas can be administered for, for example, 30 minutes to one hour, one hour to two hours, two hours to three hours, or longer. Additionally, when a gas containing molecular hydrogen is administered in a transpulmonary manner by inhalation or suction, the gas can be administered to subjects under an atmospheric pressure environment, or, for example, under a high atmospheric pressure in the range exceeding a standard atmospheric pressure (i.e., approximately 1.013 atm) and not higher than 7.0 atm, for example, under a high atmospheric pressure environment in the range of 1.02 to 7.0 atm, preferably in the range of 1.02 to 5.0 atm, more preferably in the range of 1.02 to 4.0 atm, yet more preferably in the range of 1.02 to 1.35 atm (including the gas containing molecular hydrogen).

### 2. A Method for Preventing and/or Improving Encephalitis and/or Meningitis and a Symptom Associated with the Encephalitis and/or Meningitis

The composition containing molecular hydrogen, encephalitis and/or meningitis and a symptom associated with the encephalitis and/or meningitis, dose, administration method, and the like are as described in the above 1.

In the method of the present invention, a gas containing molecular hydrogen (preferably, air or oxygen) at higher than zero (0) and not higher than 18.5% by volume, for example, 0.5% to 18.5% by volume, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, or the like, preferably 5% to 10% by volume, 5% to 8% by volume, for example, 6% to 10% by volume, 6% to 8% by volume, 6% to 7% by volume, or the like can be inhaled or sucked by subjects for, for example, one to three hours or longer per day and can be continued for, for example, one to three months or longer, four to seven months or longer, one to three years or longer.

Or, in the method of the present invention, for example, 200 to 500 mL per dose for intravenous administration or, for example, 500 to 1000 mL per dose for oral administration of a liquid containing molecular hydrogen at a concentration of, for example, 1 to 10 ppm, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, 5 to 7 ppm, or the like, preferably 3 to 10 ppm, 4 to 10 ppm, 5 to 10 ppm, 5 to 9 ppm, 5 to 8 ppm, 5 to 7 ppm, or the like can continue to be administered to subjects for, for example, 0.5 to three months or longer, four to seven months or longer, one to three years or longer.

The method of the present invention may further be used in combination with a therapeutic agent used for the treatment of encephalitis and/or meningitis and a symptom associated with the encephalitis and/or meningitis, if necessary. Such a combination use is expected to increase levels of prevention and/or improvement of encephalitis and/or meningitis and a symptom associated with the encephalitis and/or meningitis.

### EXAMPLE

The present invention is explained more specifically with reference to the following example. However, the example is not intended to limit the scope of the present invention.

### Example 1

### A Case of Encephalitis and/or Meningitis and a Symptom Associated with the Encephalitis and/or Meningitis Improved by Hydrogen Inhalation

The case patient is 65-year-old male. The patient presenting with pyrexia and disturbance of consciousness was admitted to hospital urgently. Meningococci were identified in cultured blood and cerebrospinal fluid, and meningococcal meningitis was diagnosed. The brain MRI showed observations consistent with ventriculitis. Ceftriaxone is administered to the patient for 17 days, which resolved symptoms, but he experienced relapse after completion of the administration. The patient initiated inhalation, which was performed for one to three hours per day for one month using a hydrogen gas generator “Jobs-α” (a hydrogen concentration, 4% to 6%; generation rate of hydrogen gas, 200 mL/min) manufactured by MiZ Company Limited. Then, the clinical course began to improve, and the exam of the brain MRI confirmed the improvement of ventriculitis.

The present invention can prevent and/or improve encephalitis and/or meningitis and a symptom associated with the encephalitis and/or meningitis by administering a composition containing molecular hydrogen.

What is claimed is:

1. A method for preventing and/or improving encephalitis, meningitis, or a symptom associated with encephalitis or meningitis in a subject, comprising administering to the subject a composition comprising an effective amount of molecular hydrogen.

2. The method according to claim 1, wherein the encephalitis or meningitis is a disease selected from the group consisting of bacterial meningitis, tuberculous meningitis, fungal meningitis, viral meningitis, meningitis due to a spread of cancerous inflammation, and herpes encephalitis.

3. The method according to claim 1, wherein the symptom associated with the encephalitis or meningitis is a symptom selected from the group consisting of headache, pyrexia, disturbance of consciousness, stiffness of the neck, vomiting, visual impairment, convulsion, memory disturbance, personality change, numbness, and abnormal behavior.

4. The method according to claim 1, wherein the composition is a liquid composition or a gas composition.

5. The method according to claim 4, wherein the liquid composition has a hydrogen concentration of 1 to 10 ppm.

6. The method according to claim 4, wherein the gas composition has a hydrogen concentration of higher than zero (0) and not higher than 18.5% by volume.

7. The method according to claim 1, wherein the subject is a mammalian subject.

8. The method according to claim 1, wherein the subject is a human.

9. The method according to claim 1, wherein the composition is produced by using a hydrogen gas generating apparatus, a hydrogen water generating apparatus, or a hydrogen gas adding apparatus.
